(19) United States
(12) Patent Application Publication
(10) Pub. No.: US 2017/0049668 A1
(43) Pub. Date: Feb. 23, 2017

(54) CLEANSING COMPOSITION WITH VISUAL PARTICLES

(71) Applicant: The Dial Corporation, Scottsdale, AZ (US)

(72) Inventors: James A. Fernandez, Phoenix, AZ (US); Isil Kayiran, Seattle, WA (US); Mary J. Conway, Phoenix, AZ (US); Jeffrey M. Rosenfeld, Phoenix, AZ (US)

(21) Appl. No.: 14/829,870

(22) Filed: Aug. 19, 2015

(51) Int. Cl. A61K 8/04; A61Q 19/10; B65D 25/38; A61K 8/44

(52) U.S. Cl. CPC A61K 8/044; A61K 8/442; A61Q 19/10; B65D 25/38; A61K 2800/87

(57) ABSTRACT

A composition and a consumer product are provided for cleansing the skin. The composition includes, in a cosmetically suitable carrier, at least one surfactant and at least one solid particle. The composition is dispensed as a foam. The consumer product includes a container and a cleansing composition. The cleansing composition includes a cosmetically suitable carrier, at least one surfactant, and at least one solid particle.

### FIELD OF THE INVENTION

The present invention generally relates to cleansing compositions, and more particularly relates to cleansing compositions containing solid particles.

### BACKGROUND OF THE INVENTION

Humans have been making cleansing compositions since the times of ancient Babylon. In the almost five millennia since humans started making cleansing compositions, the compositions have taken a variety of forms, including both solid and liquid cleansing compositions.

Liquid cleansing compositions have gained much popularity because the liquid form of a cleansing composition allows incorporation of at least one additive ingredient that improves the rheological properties, antibacterial efficacy, foam generation, and/or other properties which cause liquid cleansing compositions to appeal to consumers. Liquid cleansing compositions can be provided in dispensers that provide a consumer with the impression that the liquid cleansing composition is not readily contaminated.

Certain liquid cleansing compositions have incorporated solid particles, which may be able to function as exfoliants, or substances which abrade the outer layer of skin and allow the cleansing composition—including skin benefit agents, such as moisturizers—to access a lower layer of skin cells. In some cases, this provides for skin that is more effectively moisturized, and which appears healthier and more vibrant. Liquid cleansing compositions that include solid particles may also include a high concentration of a suspending polymer, which immobilizes the solid particles, such that the solid particles remain evenly dispersed throughout the solution. However, some suspending polymers cause skin irritation on certain types of skin. The incorporation of solid particles may also appeal to consumers due to the pleasant visual appearance of the solid particles in the composition.

However, solid particles have not been incorporated into foaming compositions, which may be due to technical difficulties. For example, the solid particles may obstruct the foam generator, which would decrease the efficiency of foam generation or clog the dispenser. Additionally, foaming compositions have a lower viscosity than non-foaming liquid cleansing compositions because the generation of a foam increases the viscosity of the dispensed composition, which may cause foaming cleansing compositions to be incompatible with the high concentration of suspending polymers that allows the solid particles to remain dispersed throughout the solution.

Accordingly, it is desirable to provide a foaming cleansing composition that contains solid particles. In addition, it is desirable to provide a cleansing composition that contains solid particles and has a low concentration of a suspending polymer. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A composition is provided for cleansing skin. The composition includes, in a cosmetically suitable carrier, at least one surfactant and at least one solid particle that is visible to the human eye without the aid of a magnifying lens or optical device. The cleansing composition is dispensed as a foam.

A consumer product is provided for cleansing skin. The consumer product includes a container and a cleansing composition. The cleansing composition includes a cosmetically suitable carrier, at least one surfactant, and at least one solid particle. The cleansing composition has a viscosity, excluding the solid particles, of between 0 and 3,000 centipoise (cps). The at least one solid particle sinks to a bottom of the container when the container is at rest.

A consumer product is provided for cleansing skin. The consumer product includes a transparent plastic container that includes a dispenser unit and a cleansing composition. The cleansing composition includes a cosmetically suitable carrier, at least one surfactant, including a zwitterionic surfactant, and at least one solid particle. The cleansing composition is dispensed from the transparent plastic container in the form of a foam that contains at least 50% by volume of gas. The solid particles are sparingly soluble in the cleansing composition, and have a specific gravity, relative to the cosmetically suitable carrier, of greater than or equal to 1.

### BRIEF DESCRIPTION OF THE DRAWINGS

The present invention will hereinafter be described in conjunction with the following drawing figures, wherein like numerals denote like elements, and

FIG. 1 is a diagram of a consumer product containing a cleansing composition which includes solid particles in a settled state, according to an example of the principles described herein; and

FIG. 2 is a diagram of a consumer product containing a cleansing composition that includes solid particles in a dispersed state, according to an example of the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Due to technical difficulties, solid particles have not been incorporated into foaming cleansing compositions. For example, solid particles may clog a mesh that is used in foam generation, which could cause the dispenser to be less efficient, or to be unable to dispense the foaming cleansing composition altogether. Additionally, foaming compositions may be incompatible with concentrations of suspending polymers that are able to suspend solid particles.

However, including solid particles in a cleansing composition may provide consumer appeal, as the solid particles can be redistributed throughout the cleansing composition by agitation, which could cause a swirling and enticing visual effect. For example, solid particles which fall slowly in a liquid cleansing composition provide a pleasing visual effect. Agitation by shaking or inversion of the container can cause the solid particles to disperse within the liquid cleansing composition, which provides a visual effect as the particles descend to the bottom of the container. Such a visual effect may provide consumer appeal, and provides an appearance that is akin to falling snow.

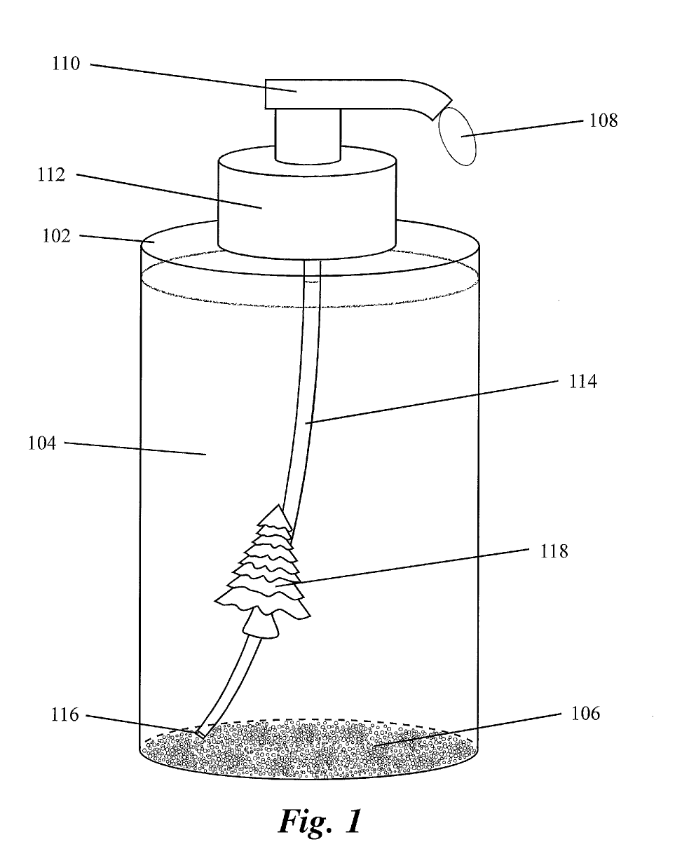

Turning now to the figures, FIG. 1 is a diagram of a consumer product containing a cleansing composition that includes solid particles in a settled state, according to an example of the principles described herein. The consumer product includes a container (102), which holds the liquid cleansing composition (104). The liquid cleansing composition (104) includes solid particles (106); as depicted in FIG. 1, the solid particles (106) have settled to the bottom of the container (102). The liquid cleansing composition (104) can be dispensed as a foam (108). The foam (108) may be generated upon actuation of a lever (110). The generation of the foam (108) occurs in a foam generation chamber (112), which may include at least one mesh screen, or may operate by another mechanism. The foam generation chamber (112) may operate by drawing the liquid cleansing composition (104) through a dip tube (114) into the foam generation chamber (112); the actuation of the lever (110) causes air from the environment to pass through a mesh screen in the presence of the liquid cleansing composition (104), causing bubbles to form in the liquid cleansing composition (104), such that the liquid cleansing composition (104) takes the form of a foam (108), which is ejected from an opening in the lever (110). The dip tube (114) may be equipped with a pre-filter (116). The dip tube may also be equipped with a pendant (118), which could enhance a visual effect.

In one example, the dip tube (114) may also include a pre-filter (116), which is a screen with openings such that the solid particles (106) are not taken up into the dip tube (114), and thus do not clog the mesh screen of the foam generation chamber (112). In such an example, the solid particles (106) may be included in order to provide a visual effect. In another example, the dip tube (114) does not include a pre-filter (116), and the particle size of the solid particles (106) and the arrangement of the at least one mesh screen used for foam generation in the foam generation chamber (112) are chosen so that the solid particles (106) pass through the mesh screens without obstructing the flow of gas through the mesh. In a further example, the foam generation chamber (112) operates without the use of a mesh, which allows the solid particles (106) to pass through the foam generation chamber (112) without obstructing the generation, or dispensing, of a foam (108). Such alternatives to mesh may include, for example, the use of a fibrous material, a porous substrate, a three-dimensional network of solid material, or the motion of an internal element of the foam generation chamber (112), which motion may be caused by actuation of the lever (110).

A liquid cleansing composition (104) according to the present specification includes at least one surfactant. A surfactant has at least one hydrophobic end and at least one hydrophilic end. The at least one hydrophobic end allows the surfactant to interact with uncharged molecules, such as oils. The at least one hydrophilic end facilitates the interaction of the molecule with charged or polar molecules, such as water. The at least one hydrophilic end can be used to classify surfactants, which may be anionic, cationic, nonionic, amphoteric, or zwitterionic. Anionic surfactants have a negatively charged hydrophilic end. Examples of anionic surfactants include sulfate, sulfonate, carboxylate, phosphate, or the like. Anionic surfactants may be sensitive to water hardness. Cationic surfactants have a positively charged hydrophilic end, such as a quaternary amine. Nonionic surfactants have a hydrophilic end that is charge neutral, such as an ethoxylate, glycoside, or poly-ol; such surfactants may not be sensitive to water hardness. Amphoteric surfactants have a hydrophilic end that has a functional group that is capable of acting as a base, and a functional group that is capable of acting as an acid, such as amine oxides. Zwitterionic surfactants have both a positive and negative charge on their hydrophilic ends, such as sultaines, or betaines. The hydrophobic end may include a saturated or unsaturated, linear or branched, substituted or unsubstituted, cyclic or acyclic alkyl chain containing at least 8 carbon atoms.

For the purposes of the present specification, “alkyl” refers to saturated or unsaturated, branched or unbranched, cyclic or acyclic, substituted or unsubstituted hydrocarbon chains of any length. For example, alkyl may refer to saturated hydrocarbon chains, such as lauryl groups (—C₁₂H₂₅), myristyl groups (—C₁₄H₂₉), cetyl groups (—C₁₆H₃₃), stearyl groups (—C₁₈H₃₇), isostearyl groups (—C₁₈H₃₇), and the like. In another example, alkyl may refer to unsaturated hydrocarbon chains, such as oleyl groups (—C₁₈H₃₅), linoleyl groups (—C₁₈H₃₃), and the like. In a further example, alkyl may refer to hydrocarbons bearing additional heteroatom substituents, such as ricinoleyl groups (C₁₈H₃₅O), and the like. In a still further example, alkyl may refer to hydrocarbons bearing cyclic groups, which may contain heteroatoms, such as dodecylbenzyl groups (—C₁₈H₂₉), dodecylpyridinyl groups (—C₁₇H₂₉N), and the like.

Anionic surfactants include alkyl carboxylic acids, alkyl ether carboxylic acids, alkyl phosphates, alkyl ether phosphates, alkyl sulfates, alkyl ether sulfates, alkyl sulfonates, alkyl ether sulfonates, and salts thereof. In principle, any type, number, or combination of anionic surfactants may be used in compositions according to the present specification; selected examples are provided below. It is also possible for a liquid cleansing composition (104) according to the present specification to include other types of surfactants, without including anionic surfactants.

Alkyl carboxylic acids are one type of anionic surfactant. Alkyl carboxylic acids contain an alkyl group that is directly connected to a carboxylic acid group. Carboxylic acids may be provided in either their acid form (—COOH), or as salts of alkali metals, alkaline earth metals, ammonium, or combinations thereof. For example, potassium salts of alkyl carboxylic acids may be suitable for use in a liquid cleansing composition (104) according to the present specification.

Non-limiting examples of alkyl carboxylic acids which may be included in a liquid cleansing composition (104) according to the present specification include lauric acid, myristic acid, palmitic acid, stearic acid, behenic acid, oleic acid, palmitoleic acid, linoleic acid, linolenic acid, isostearic acid, ricinoleic acid, hydroxystearic acid, dihydroxystearic acid, and salts thereof.

Further non-limiting examples of alkyl carboxylic acids which may be included in a liquid cleansing composition (104) according to the present specification include carboxylic acids or salts thereof that are prepared from triglycerides by hydrolysis, and may be used without purification to homogeneity. The hydrolysis of triglycerides may be either alkaline or non-alkaline, and may produce either soaps (salts of alkyl carboxylic acids) or free fatty acids (the acid form). The hydrolysis products may be mixtures of alkyl carboxylic acids, which may be used directly. Such mixtures are, for example, coconut oil fatty acid, palm kernel oil fatty acid, tallow fatty acid, hydrogenated tallow fatty acid, technical-grade oleic acid, technical-grade palmitic/stearic acid, and soybean oil fatty acid.

Another type of anionic surfactant that may be used in a liquid cleansing composition (104) according to the present specification is the alkyl sulfate type of surfactant. Alkyl sulfates may also be used according to the present specification as salts of ammonia, alkali or alkaline earth metals.

Suitable non-limiting examples of alkyl sulfates which may be used in a liquid cleansing composition (104) according to the present specification include lauryl sulfate, cetyl sulfate, stearyl sulfate, oleyl sulfate, linoleyl sulfate, and salts and/or mixtures thereof. For example, sodium, potassium or ammonium salts of the above alkyl sulfates may be used.

A further type of anionic surfactant that may be used in a liquid cleansing composition (104) according to the present specification is the alkyl ether sulfate type of surfactant, which are surface-active agents that contain an ether linkage separating the alkyl group from the sulfate group. In principle, any ether linkage of a diol or polyol may be used. In one example, ethylene or polyethylene glycol ether linkages are used. In another example, propylene or polypropylene glycol ethers are used. In a further example, glyceryl or polyglyceryl ether linkages are used. In a still further example, butylene or polybutylene glycol ethers are used. Alkyl ether sulfates may be provided in either the protonated form or as salts of ammonia, alkali or alkaline earth metals.

Suitable non-limiting examples of alkyl ether sulfates which may be used according to an example of the present specification include laureth-2 sulfate, laureth-3 sulfate, myreth-2 sulfate, myreth-5 sulfate, ceteth-2 sulfate, ceteth-4 sulfate, steareth-2 sulfate, steareth-3 sulfate, steareth-4 sulfate, and mixtures and/or salts thereof.

Another type of anionic surfactant that may be used in a liquid cleansing composition (104) according to the present specification is an alkyl ether carboxylic acid type of surfactant. Alkyl ether carboxylic acids are surface-active agents that have a carboxylic group as the hydrophilic group, and an ether linkage between the carboxylic acid group and the alkyl chain. In principle, any ether linkage of a diol or polyol may be used. In one example, ethylene or polyethylene glycol ether linkages are used. In another example, propylene or polypropylene glycol ethers are used. In a further example, glyceryl or polyglyceryl ether linkages are used. In a still further example, butylene or polybutylene glycol ethers are used. Alkyl ether carboxylic acids may be provided in either the protonated (acid) form, or as salts of ammonia, alkali or alkaline earth metals.

Suitable non-limiting examples of alkyl ether carboxylic acids include ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, and mixtures and/or salts thereof.

Another type of anionic surfactant that may be used in a liquid cleansing composition (104) according to the present specification is an alkyl sulfonate type of surfactant. Alkyl sulfonates are surface-active agents that have an alkyl group directly linked to the sulfur of the sulfonate group. Alkyl sulfonates may also be provided as salts of ammonia, alkali or alkaline earth metals.

Suitable non-limiting examples of alkyl sulfonate type surfactants which may be used in a liquid cleansing composition (104) according to an example of the present specification include dodecylbenzenesulfonate, C13-17 alkane sulfonate, C14-18 alkane sulfonate, cocoamphohydroxypropylsulfonate, C12-14 olefin sulfonate, C14-16 olefin sulfonate, C16-18 olefin sulfonate, and mixtures and/or salts thereof.

A further type of anionic surfactant that may be used in a liquid cleansing composition (104) according to the present specification is an alkyl phosphate type of surfactant. Alkyl phosphates are surfactants that contain a phosphate group as the hydrophilic group, and contain at least one alkyl group. Alkyl phosphates may be provided in either the protonated form or as an ammonia, alkali or alkaline earth metal salt.

Suitable non-limiting examples of alkyl phosphate surfactants which may be used in liquid cleansing compositions (104) according to an example of the present specification include phospholipid EFA (linoleamidopropyl PG-dimonium chloride phosphate), phospholipid PTC (cocamidopropyl PG-dimonium chloride phosphate), lauryl phosphate, dilauryl phosphate, myristyl phosphate, cetyl phosphate, dicetyl phosphate, stearyl phosphate, distearyl phosphate, oleyl phosphate, and salts and/or mixtures thereof.

A still further type of anionic surfactant that may be used in a liquid cleansing composition (104) according to the present specification is an alkyl ether phosphate type of surfactant. Alkyl ether phosphates are surface-active agents that contain an ether linkage between one or more alkyl groups and the phosphate group. In principle, any ether linkage of a diol or polyol may be used. In one example, ethylene or polyethylene glycol ether linkages are used. In another example, propylene or polypropylene glycol ethers are used. In a further example, glyceryl or polyglyceryl ether linkages are used. In a still further example, butylene or polybutylene glycol ethers are used. Alkyl ether phosphates may be provided in either their protonated form or as an ammonia, alkali or alkaline earth metal salt.

Suitable non-limiting examples of alkyl ether phosphates include PPG-5-ceteth-10 phosphate, oleth-3 phosphate, oleth-10 phosphate, and mixtures and/or salts thereof.

Anionic surfactants may be included in a liquid cleansing composition (104) according to the present specification in amounts ranging from 0% to 50% by weight. In one example, a concentration of anionic surfactants in a liquid cleansing composition (104) according to the present specification ranges from 5% to 35%. In a further example, a concentration of anionic surfactants in a liquid cleansing composition (104) according to the present specification ranges from 15% to 25%. All of the above concentrations are provided as weight percentages, relative to the total weight of the composition.

A liquid cleansing composition (104) according to the present specification may also contain a nonionic surfactant. For example, the composition may include alkoxylated fatty alcohols, alkoxylated fatty esters, alkanolamides, alkyl glycosides, and combinations thereof. It is also possible for a liquid cleansing composition (104) according to the present specification to include other types of surfactants, without including nonionic surfactants.

Alkoxylated fatty alcohols may be incorporated into a liquid cleansing composition (104) according to the present specification. Alkoxylated fatty alcohols are condensation products of at least one alkoxy group with a fatty alcohol. In principle, any type or number of alkoxy groups may be used. In one example, ethylene glycol or polyethylene glycol, propylene or polypropylene glycol, glyceryl or polyglyceryl, butylene or polybutylene glycol are used. In a further example, combinations of the above are used, such as a combination of polyethylene glycol and polypropylene glycol, as either a random- or block-condensation product.

Suitable non-limiting examples of alkoxylated fatty alcohols for use in a liquid cleansing composition (104) according to the present specification are ceteth-10, ceteth-15, ceteth-20, ceteth-25, steareth-2, steareth-10, steareth-20, steareth-25, steareth-100, laureth-10, trideceth-10, PEG-4-PPG-7 C13/15 alcohol, PPG-8-ceteth-5, PPG-4-laureth-5, and combinations thereof.

Another type of nonionic surfactant that may be used in a liquid cleansing composition (104) according to the present specification is an alkoxylated fatty ester type of surfactant. Alkoxylated fatty esters are esters of fatty acids with one or more alkoxy groups. In principle, any type or number of alkoxy groups may be used. In one example, ethylene glycol or polyethylene glycol, propylene or polypropylene glycol, glyceryl or polyglyceryl, butylene or polybutylene glycol are used. In a further example, combinations of the above may be used, such as a combination of glyceryl and polyethylene glycol.

Suitable non-limiting examples of alkoxylated fatty esters include glyceryl stearate, glyceryl cetate, PEG-2 laurate, PEG-10 laurate, PEG-20 laurate, PEG-2-PPG-5 laurate, PEG-2 stearate, PEG-20 stearate, PPG-15 isostearate, PPG-15 stearate, PEG-3 glyceryl cocoate, PEG-7 glyceryl cocoate, PEG-30 glyceryl cocoate, PEG-3 glyceryl isostearate, PEG-6 glyceryl isostearate, PEG-6 glyceryl triisostearate, PEG-5 glyceryl trioleate, and the like.

Another type of nonionic surfactant that may be used in a liquid cleansing composition (104) according to the present specification is an alkanolamide type of surfactant.

Suitable non-limiting examples of alkanolamides which may be used in a liquid cleansing composition (104) according to the present specification include coco monoethanolamide (MEA), coco diethanolamide (DEA), lauryl MEA, lauryl DEA, myristyl MEA, myristyl DEA, cetyl MEA, cetyl DEA, stearyl MEA, stearyl DEA, oleyl MEA, oleyl DEA, and combinations thereof.

Another type of nonionic surfactant that may be used in a liquid cleansing composition (104) according to the present specification is an alkyl glycoside type of surfactant. Alkyl glycosides are condensation products of fatty alcohols with at least one sugar. While each molecule of an alkyl glycoside contains an integer number of sugars, the ensemble average may be a noninteger number. For example, an alkyl glycoside surfactant could contain glucose as the sugar, and could be included as a mixture of compounds with 2, 3, 4 and 5 glucose units, such that the ensemble average contains 3.4 glucose units.

Suitable non-limiting examples of alkyl glycosides that may be used in a liquid cleansing composition (104) according to the present specification include coco glucoside and lauryl gluco side.

Nonionic surfactants may be incorporated into a liquid cleansing composition (104) according to the present specification at concentrations ranging from 0% to 50% by weight. In one example, a liquid cleansing composition (104) according to the present specification contains at least one nonionic surfactant at a concentration ranging from 0.01% to 15% by weight. In another example, a liquid cleansing composition (104) according to the present specification contains at least one nonionic surfactant at a concentration ranging from 0.1% to 10% by weight. In a further example, a liquid cleansing composition (104) according to the present specification contains a nonionic surfactant at a concentration ranging from 0.1% to 5% by weight. All of the above concentrations are provided as weight percentages, relative to the total weight of the composition.

A liquid cleansing composition (104) according to the present specification may include cationic surfactants. Cationic surfactants include any surfactant that contains a positive charge and does not contain a negative charge, such as, for example, quaternary ammonium salt surfactants and tertiary ammonia surfactants (which forms a quaternary ammonium surfactant in compositions with a pH of less than about 9). Additional types of cationic surfactants that may be used in a liquid cleansing composition (104) according to the present specification include esterquat and amidoamine surfactants. It is also possible for a liquid cleansing composition (104) according to the present specification to include other types of surfactants, without including cationic surfactants.

Suitable non-limiting examples of cationic surfactants which may be used in a liquid cleansing composition (104) according to the present specification include cetrimonium chloride, steartrimonium chloride, behentrimonium chloride, arachidtrimonium chloride, stearamidopropyl trimonium chloride, N,N-dimethylstearylamine, N,N-dimethylcetylamine, lauramidopropyl PG-dimonium chloride, cocamidopropyl PG-dimonium chloride, ditallowoyl PG-dimonium chloride, oleamidopropyl PG-dimonium chloride, isostearamidopropyl PG-dimonium chloride, behenamidopropyl PG-dimonium chloride, and the like. While each of the aforementioned examples employs chlorine salts, it may also be suitable to use salts formed with other anions, for example, bromine or methosulfate.

Cationic surfactants may be incorporated in a liquid cleansing composition (104) according to the present specification in concentrations ranging from 0% to 50% by weight. In one example, a liquid cleansing composition (104) according to the present specification contains from 0.01% to 15% by weight of a cationic surfactant. In another example, cationic surfactants are included in a liquid cleansing composition (104) according to the present specification at concentrations ranging from 0.1% to 10% by weight. In a further example, cationic surfactants are included in a liquid cleansing composition (104) according to the present specification at concentrations ranging from 1% to 5% by weight. A still further example contains cationic surfactants in a liquid cleansing composition (104) according to the present specification at a concentration ranging form 0.1% to 2% by weight, with all weights being relative to the total weight of the composition.

A liquid cleansing composition (104) according to the present specification may also include amphoteric surfactants, which are surfactants that have a hydrophilic part which has both acidic and basic hydrophilic groups and which behaves in an acidic or basic manner, depending on the conditions. Unlike zwitterionic surfactants, amphoteric surfactants do not permanently bear a charge. Amphoteric surfactants include surfactants based on aliphatic amines having carboxy, sulfo or phosphono side chains. Amphoteric surfactants include such surfactants as N-alkyl glycines, N-alkyl propionic acids, N-alkyl aminobutyric acids, N-alkyl taurines, N-alkyl sarcosines, and amine oxide surfactants. It is also possible for a liquid cleansing composition (104) according to the present specification to include other types of surfactants, without including amphoteric surfactants.

Suitable non-limiting examples of amphoteric surfactants which may be included in a liquid cleansing composition (104) according to the present specification include lauryldimethylamine oxide (lauramine oxide), laurylamidopropyldimethylamine oxide (lauramidopropylamine oxide), myristyldimethylamine oxide (myristamine oxide), cetyldimethylamine oxide (palmitamine oxide, cetamine oxide), stearyldimethylamine oxide (stearamine oxide), stearamidopropyldimethylamine oxide (stearamidopropylamine oxide), isostearamidopropylamine oxide, cocamine oxide, oleamine oxide, oleamidopropylamine oxide, dihydroxyethyl lauramine oxide, dihydroxyethyl cocamine oxide, dihydroxyethyl stearamine oxide, PEG-3 lauramine oxide, disodium cocoamphodiacetate, sodium cocoamphoacetate, disodium cocoamphodipropionate, disodium lauroamphodiacetate, sodium lauramphoacetate, disodium stearoamphodiacetate, sodium stearoamphoacetate, sodium isostearamphoacetate, disodium isostearoamphodipropionate, and combinations thereof.

Amphoteric surfactants may be included in a liquid cleansing composition (104) according to the present specification at concentrations ranging from 0% to 50% by weight. In one example, a liquid cleansing composition (104) according to the present specification contains at least one amphoteric surfactant at a concentration ranging from 0.01% to 25% by weight. In another example, a liquid cleansing composition (104) according to the present specification contains at least one amphoteric surfactant at a concentration ranging from 0.1% to 15% by weight. In a further example, at least one amphoteric surfactant is present in a liquid cleansing composition (104) according to the present specification at a concentration ranging from 1% to 10% by weight. In a still further example, at least one amphoteric surfactant is present in a liquid cleansing composition (104) according to the present specification at a concentration ranging from 0.1% to 5% by weight, with all weights being relative to the total weight of the composition.

A liquid cleansing composition (104) according to the present specification may also include zwitterionic surfactants. Zwitterionic surfactants are surfactants that bear both a positive charge and a negative charge. Some zwitterionic surfactants are capable of forming intramolecular salts. Zwitterionic surfactants include the betaine type of surfactants as well as the sultaine type of surfactants. It is also possible for a liquid cleansing composition (104) according to the present specification to include other types of surfactants, without including zwitterionic surfactants.

Suitable non-limiting examples of zwitterionic surfactants which may be included in a liquid cleansing composition (104) according to the present specification include cocamidopropyl betaine, cocamidopropyl sultaine, cocamidopropyl hydroxysultaine, laurylamidopropyl betaine, laurylamidopropyl sultaine, stearylamidopropyl betaine, oleyl betaine, myristyl betaine, stearyl betaine, cetyl betaine, and the like. Combinations of the above are also suitable for incorporation into a liquid cleansing composition (104) according to the present specification.

Zwitterionic surfactants may be included in a liquid cleansing composition (104) according to an example of the present specification at concentrations ranging from 0% to 50% by weight. In one example, a liquid cleansing composition (104) according to the present specification contains at least one zwitterionic surfactant at a concentration ranging from 0.01% to 25% by weight. In another example, a liquid cleansing composition (104) according to the present specification contains at least one zwitterionic surfactant at a concentration ranging from 0.1% to 15% by weight. In a further example, at least one zwitterionic surfactant is present in a liquid cleansing composition (104) according to the present specification at a concentration ranging from 1% to 10% by weight. In a still further example, a zwitterionic surfactant is present in a liquid cleansing composition (104) according to the present specification at a concentration ranging from 4% to 10% by weight, with all weights being relative to the total weight of the composition.

Further types of surfactants that may be used in a liquid cleansing composition (104) according to the present specification are silicone surfactants. Silicone surfactants are surfactants which have a hydrophobic part provided by a linear or branched silicone chain, which is a chain with alternating silicon and oxygen atoms, such that the silicon atoms also contain alkyl substituents. Silicone surfactants may be anionic, cationic, nonionic, amphoteric or zwitterionic, and may be included in a liquid cleansing composition (104) according to the present specification at concentrations based on the classification of their hydrophilic head groups, at the concentrations provided above for each type of surfactant. It is also possible for a liquid cleansing composition (104) according to the present specification to include other types of surfactants, without including silicone surfactants.

Suitable non-limiting examples of silicone surfactants include dimethicone PEG-7 sulfate, dimethicone PEG-12 sulfate, dimethicone-PG diethylmonium chloride, dimethicone hydroxypropyl trimonium chloride, bis-PEG-4 dimethicone, bis-PEG-12 dimethicone, PEG-8 amodimethicone, PEG-12 dimethicone, PEG/PPG-12/16 dimethicone, PPG-12 dimethicone, and the like.

A liquid cleansing composition (104) according to the present specification also includes at least one solid particle (106). Such solid particles (106) are chosen so as to remain solid in the liquid cleansing composition (104) at ambient temperature and pressure. Accordingly, solid particles (106) according to the present specification may be sparingly soluble in liquid cleansing composition (104), or may be insoluble in the liquid cleansing composition (104). In some examples, the solid particles (106) swell when mixed with the liquid cleansing composition (104). The swelling of solid particles (106) being distinct from the dissolution of solid particles (106) in the liquid cleansing composition (104). The extent to which a type of solid particle (106) dissolves in the liquid cleansing composition (104) can be determined by mixing 10 grams of the solid particles (106) with 1 liter of the carrier of the liquid cleansing composition (104), with constant stirring at standard ambient temperature and pressure (SATP, corresponding to a temperature of 25 degrees Celsius (° C.) and a pressure of 100 kilopascals (kPa)) until an equilibrium is reached. Once an equilibrium is reached, the solution is filtered, and the remaining solid particles (106) are dried in an oven at 80° C. until their mass is constant, and the fraction of the solid particles (106) that remain in solid form is determined. The solubility of the solid particles (106) is determined in the carrier of a liquid cleansing composition (104) according to the present specification without surfactants or other additives such that these components do not leave a residue after drying that affects the final mass of the solid particles (106).

For the purposes of the present specification, solid particles (106) are said to be sparingly soluble in the liquid cleansing composition (104) if less than 20% by weight of the solid particles (106) dissolve in the liquid cleansing composition (104) at SATP. In one example, solid particles (106) are sparingly soluble in a liquid cleansing composition (104) according to the present specification if less than 10% by weight, such as less than 5% by weight of the solid particles (106) dissolves at equilibrium at SATP. In a further example, solid particles (106) are sparingly soluble in a liquid cleansing composition (104) according to the present specification if less than 2% by weight, such as less than 1% by weight of the solid particles (106) dissolves at equilibrium at SATP. Certain types of solid particles (106) may also be inert with respect to solvation in a liquid cleansing composition (104) according to the present specification. Accordingly, solid particles (106) that do not dissolve at all in a liquid cleansing composition (104) according to the present specification are said to be insoluble. Whether a type of solid particle (106) is soluble, sparingly soluble or insoluble may be a function of the carrier that is used for a liquid cleansing composition (104) according to the present specification.

Solid particles (106) according to present specification may be a variety of shapes. According to one example, solid particles (106) according to the present specification are convex particles. Convex particles are particles that curve outward. Alternatively, convex particles are particles such that the shortest path between any two points on a single particle passes through, or is tangent to the surface of, the particle. According to a further example, solid particles (106) according to the present specification are concave particles. Concave particles are particles that have an inward-curving surface. Alternatively, concave particles are particles such that there exist two points on the particle for which the shortest path between the two points is not entirely within, or tangent to, the particle. Solid particles (106) according to the present specification may also have a recognizable shape, such as, for example, spheres, hearts, stars, discs, and the like, or combinations thereof.

Solid particles (106) according to the present specification may be a variety of sizes, and have a variety of size distributions. The particle size distribution may affect the propensity for the particles to affect the efficiency of the foam generation chamber (112), or the ability of the pre-filter (116) to retain the solid particles (106) within the container (102). The size of a solid particle (106) according to the present specification is the diameter of the smallest sphere that could surround the particle, which can also be expressed as the largest distance between any two points on the surface of the particle. In principle, any particle size or any distribution of particle sizes may be suitable. According to one example, solid particles (106) according to the present specification range from 0.005 millimeter (mm) to 50 mm, such as from 0.05 mm to 40 mm. In a further example, solid particles (106) according to the present specification range from 0.5 mm to 40 mm, such as from 0.52 mm to 38 mm. In a still further example, solid particles (106) according to the present specification range from 0.005 millimeter (mm) to 2 mm. According to a further example, solid particles according to the present specification range from 0.01 mm to 1 mm, such as from 0.01 mm to 0.1 mm. In a further example, solid particles according to the present specification range from 0.02 mm to 0.05 mm. In a still further example, solid particles (106) according to the present specification have a normal distribution with a median particle diameter of 0.05 millimeter (mm) and a standard deviation of 0.02 mm. According to another example, solid particles (106) according to the present specification have a normal distribution with a median particle diameter of 0.05 mm and a standard deviation of 0.01 mm. In a further example, solid particles (106) according to the present specification have a uniform distribution of particle size, ranging from 0.01 mm to 0.1 mm. In a still further example, solid particles (106) according to the present specification have an F distribution of particle size, with a maximum at 10 micrometers (μm). Each of the aforementioned particle size distributions is a continuous distribution, although the number of solid particles (106) according to the present specification within a container (102) may be finite, and thus may be said to correspond to a continuous distribution if the distribution of particle sizes approximates the continuous distribution.

Particle size distributions may also be expressed as a percentage of solid particles (106) that fall within a range of particle sizes. In one example, solid particles (106) according to the present specification contain at least 80% by weight, such as at least 90% by weight, of particles that are between 0.001 mm and 1.0 mm. In a further example, solid particles (106) according to the present specification contain at least 70% by weight, such as at least 80% by weight, or at least 90% by weight, of particles that are between 0.01 mm and 0.1 mm. In another example, solid particles (106) according to the present specification contain at least 60% by weight, such as at least 80% by weight, of particles that are between 0.02 mm and 2 mm.

Solid particles (106) according to the present specification may have a density that is greater than or equal to the density of the liquid cleansing composition (104) without the solid particles (106). The density of the solid particles (106) does not include the space between particles, or any concave surface of the solid particles (106), and thus differs from the bulk density of the solid particles (106). The density of solid particles (106) according to the present specification is written as ρ₁₀₆, which is equal to the ratio of the mass of the solid particles (106) to the volume of the solid particles (106): m₁₀₆/V₁₀₆. Similarly, the density of a liquid cleansing composition (104) is expressed as ρ₁₀₄=m₁₀₄/V₁₀₄, and may be determined based on the liquid cleansing composition (104) with or without the solid particles (106).

Solid particles (106) according to the present specification may also have a density that is less than the density of the liquid cleansing composition (104) without the solid particles (106). Solid particles (106) according to the present specification that have a density that is less than the density of the liquid cleansing composition (104) without the solid particles float in the liquid cleansing composition (104).

The density of solid particles (106) according to the present specification may also be expressed as the specific gravity of the solid particles (106) relative to either the liquid cleansing composition (104) without the solid particles (106), or relative to the carrier of the liquid cleansing composition (104). The specific gravity of the solid particles (106) corresponds to the ratio of the density of the solid particles (106) to the density of the reference solution (which may be either the liquid cleansing composition (104) or the carrier of the liquid cleansing composition (104)). For example, a specific gravity of 2.0 would indicate that the solid particles (106) have a density that is twice the density of the reference solution, and a specific gravity of 1.0 would indicate that the solid particles (106) have a density that is equal to the density of the reference solution. Solid particles (106) that have a specific gravity of 1.00 relative to the liquid cleansing composition (104) are said to be neutrally buoyant in the liquid cleansing composition (104). Solid particles (106) that have a specific gravity of less than 1, relative to the liquid cleansing composition (104) float in the liquid cleansing composition (104). Similarly, solid particles (106) which have a specific gravity of greater than 1, relative to the liquid cleansing composition (104) sink in the liquid cleansing composition (104). The greater the deviation of the specific gravity of the solid particles (106) is from 1.0 (relative to the liquid cleansing composition (104)), the greater the buoyant/sinking force the solid particles (106) will experience in the liquid cleansing composition (104). According to one example, the solid particles (106) have a specific gravity, relative to the cosmetically suitable carrier, of greater than 1, such as greater than 1.1. According to another example, the solid particles (106) have a specific gravity, relative to the cosmetically suitable carrier, of greater than 1.05, such as greater than 1.2. In a still further example, solid particles (106) according to the present specification have a specific gravity, relative to the cosmetically suitable carrier, of greater than 1.15, for example, greater than 1.25. According to a still further example, the solid particles (106) have a specific gravity, relative to the cosmetically suitable carrier, of less than 1, such as less than 0.95. In yet another example, the solid particles (106) have a specific gravity, relative to the cosmetically suitable carrier, of less than 0.9, such as less than 0.85.

According to another example, the solid particles (106) have a specific gravity, relative to the liquid cleansing composition (104) without the solid particles (106), of greater than 1, such as greater than 1.1. According to further example, the solid particles (106) have a specific gravity, relative to the liquid cleansing composition (104) without the solid particles (106), of greater than 1.05, such as greater than 1.2. In a still further example, solid particles (106) according to the present specification have a specific gravity, relative to the liquid cleansing composition (104) without the solid particles (106), of greater than 1.15, for example, greater than 1.25. According to another example, the solid particles (106) have a specific gravity, relative to the liquid cleansing composition (104) without the solid particles (106), of less than 0.98, such as less than 0.95. In a still further example, the solid particles (106) have a specific gravity, relative to the liquid cleansing composition (104) without the solid particles (106), of less than 0.92, such as less than 0.90.

In certain examples, it is advantageous to limit the specific gravity of the solid particles (106) according to the present specification, relative to either the cosmetically suitable carrier or the liquid cleansing composition (104) without the solid particles (106), in order to provide a pleasant or swirling visual effect when the container (102) is agitated. In one example, solid particles (106) according to the present specification have a specific gravity that is less than 3.0, such as less than 2.0, relative to either the cosmetically suitable carrier or the liquid cleansing composition (104) without the solid particles (106). In a further example, solid particles (106) according to the present specification have a specific gravity that is less than 1.8, such as less than 1.7, relative to either the cosmetically suitable carrier or the liquid cleansing composition (104) without the solid particles (106). In a still further example, solid particles (106) according to the present specification have a specific gravity that is less than 1.6, such as less than 1.5, relative to either the cosmetically suitable carrier or the liquid cleansing composition (104) without the solid particles (106).

Solid particles (106) according to the present specification may also be a variety of colors, or produce a variety of visual effects. In principle, any color or visual effect is suitable. For example, solid particles (106) according to the present specification may be white, red, orange, pink, blue, green, black, copper, silver, gold, goniochromatic, having a metallic reflection of incident light, or combinations thereof. The color of solid particles (106) according to the present specification may be provided by solid particles (106) that are composed of colored materials, by application of a colored coating to solid particles (106), or combinations thereof. Color may be provided by either organic or inorganic materials, or combinations thereof. In one example, color is provided to solid particles (106) according to the present specification using a dye, such as a vat dye, a material with a characteristic interaction with light, such as mica, or a pigment, such as titanium dioxide, ultramarines, or iron oxides.

Solid particles (106) according to the present specification may be composed of a variety of materials. In principle, solid particles (106) according to the present specification may be composed of any suitable material. In some examples, solid particles (106) according to the present specification are natural, synthetic or semi-synthetic polymers or copolymers. In further examples, solid particles (106) according to the present specification are either inorganic or organic materials, or combinations thereof.

Examples of suitable materials that may be used in the solid particles (106) according to the present specification include minerals or mineral-like materials, such as silicates (including glass, phyllosilicate minerals, et cetera), oxides, sulfides, sulfates, carbonates, phosphates, and mineraloids (including mother of pearl, amber, et cetera). For the purposes of the present specification, “mineral” includes substances that are conventionally considered minerals, as well as synthetically prepared equivalents of conventional minerals, substances that occur naturally but are not formed by geologic processes, and substances that are crystalline, non-crystalline or combinations thereof. In one example, inorganic pigments prepared by applying a metal oxide coating to a substrate, such as silica or mica, are used as solid particles (106) according to the present specification.

Further suitable materials that may be used in the solid particles (106) according to the present specification include biologically produced materials, such as plant matter, which have been processed to provide particulate materials. For example, actinidia chinensis (kiwi) seed, avena sativa (oat) bran, avena sativa (oat) kernel meal, carya illinoensis (pecan) shell powder, cocos nucifera (coconut) shell powder, egg shell powder, fragaria vesca (strawberry) seed, persea gratissima (avocado) fruit powder, triticum vulgare (wheat) starch, and combinations thereof. Additional natural materials that may be used in the solid particles include cellulose, mannitol, and combinations and derivatives thereof.

Still further suitable materials that may be used in the solid particles (106) according to the present specification include synthetic materials, such as polymers. Polymers include homopolymers or copolymers. Certain types of suitable polymers are crosslinked, while other suitable polymers are linear; the degree of crosslinking may be variable. Suitable examples of polymeric materials which may be used for the solid particles (106) include fumed silica, fumed alumina, cellulose derivatives, polyisoprene, proteins, protein derivatives, acrylates copolymer, polybutylene terephthalate, ethylene/vinyl acetate copolymer, plastics, including polypropylene (PP), oriented polypropylene (OPP), polyethylene terephthalate (PET), polyethylene terephthalate glycol (PETG), and biaxially oriented polypropylene (BOPP), and combinations thereof.

The solid particles (106) may be included in a liquid cleansing composition (104) according to the present specification in an amount up to 75% by weight. In one example, a liquid cleansing composition (104) according to the present specification includes from 0.001% to 50% by weight of solid particles (106). In another example, a liquid cleansing composition (104) according to the present specification includes from 0.01% to 10% of solid particles (106). In yet another example, a liquid cleansing composition (104) according to the present specification includes from 0.10% to 2.0% of solid particles (106). In a still further example, a liquid cleansing composition (104) according to the present specification includes from 0.01% to 25% by weight, such as from 0.5% to 5% by weight, or from 1% to 4% by weight of solid particles (106). In yet another example, a liquid cleansing composition (104) according to the present specification includes from 1.5% to 2.5% by weight of solid particles (106).

A liquid cleansing composition (104) according to the present specification may also contain at least one additive ingredient. Examples of additive ingredients include a natural polymer, a synthetic polymer, a semi-synthetic polymer, an antibacterial agent, a fragrance, a foaming agent, a humectant, an emollient, a dye, a pigment, a conditioning agent, a plant extract, plant matter, an essential oil, an oil, a wax, a silicone oil, a silicone wax, a chelator, a vitamin, an alkali metal halide, a preservative, and combinations thereof. The inclusion of a component that is already listed above as an additive refers to the inclusion of an additional compound within the provided class. A brief, non-limiting description of some examples of additives follows.

Natural polymers are polymers that are assembled enzymatically as a natural result of biological processes. Natural polymers may be added to an example of a liquid cleansing composition (104) according to the present specification as thickeners, natural conditioners, emulsion stabilizers, or to confer other such properties to a liquid cleansing composition (104). Natural polymers are extracted from natural sources. Non-limiting examples of natural polymers include sugar-based polymers (for example, cellulose), polymers of modified sugar units such as nucleic acid chains, proteinaceous polymers (for example, silk, keratin, and collagen), and combinations thereof. Natural polymers may be in a variety of lengths, and remain natural polymers according to the present specification if the length of the polymer is adjusted chemically after extraction of the polymer. For example, hydrolyzed keratin is a natural polymer according to the present specification, although the hydrolysis of the keratin is carried out following extraction of the keratin from natural sources. Examples of natural polymers include honey, cellulose and xanthan gum.

Synthetic polymers are polymers that are assembled from monomeric units by synthetic processes. Synthetic polymers may be added an example of a liquid cleansing composition (104) according to the present specification as thickeners, conditioners, emulsion stabilizers, or to confer other such properties to a liquid cleansing composition (104) according to the present specification. Non-limiting examples of synthetic polymers include carbomer, acrylates/acrylamide copolymers, acrylamidopropyltrimonium chloride/acrylates copolymers, polyethylene glycol polymers, polypropylene glycol polymers, polyquatemium-1, polyquaternium-2, polyquatemium-5, polyquaternium-15, polyquaternium-22, polyquaternium-28, polyquaternium-39, and combinations thereof.

Semi-synthetic polymers are polymers that involve both natural components and synthetic components. Semi-synthetic polymers may be added to an example of a liquid cleansing composition (104) according to the present specification as thickeners, conditioners, emulsion stabilizers, or to provide other such properties to a liquid cleansing composition (104) according to the present specification. Semi-synthetic polymers may be either chemically modified natural polymers or synthetically assembled polymers of natural monomer units. Non-limiting examples of semi-synthetic polymers include hydroxyethyl cellulose, methylcellulose, quatemized hydroxyethyl cellulose (which is also called polyquatemium-10), quaternized protein hydrolysates, crosslinked protein hydrolysates, and combinations thereof.

A liquid cleansing composition (104) according to the present specification may also include a thickening agent, which is a compound that increases the viscosity of the liquid cleansing composition (104). Thickening agents may be polymeric, such as natural, synthetic or semi-synthetic polymers, or may be monomeric, such as alkali halide salts. Non-limiting examples of thickening agents include methylcellulose, polyquatemium-10, hydrolyzed keratin, sodium chloride, and combinations thereof.

A liquid cleansing composition (104) according to the present specification may also include at least one antibacterial agent. An antibacterial agent is any agent that assists in the removal of bacteria, kills bacteria, or arrests bacterial growth. Some antibacterial agents also have additional functions, and belong to one or more than one of the aforementioned or following classes.

Suitable non-limiting examples of antibacterial agents include antiseptics, triclosan, benzethonium salts, benzalkonium salts, compounds which inhibit the 70S (bacterial) ribosome, and compounds which reduce the integrity of the bacterial cell wall. Additional non-limiting examples of antibacterial agents include ethanol, isopropanol, aminoglycosides (such as neomycin), cephalosporins (such as cefalexin), lincosamides (such as lincomycin), tetracyclines (such as doxycycline), penicillins (such as amoxicillin), and combinations thereof.

Fragrances are components or compositions that produce an olfactory sensation in an individual. Fragrances may contain a single component; fragrances may also be mixtures of multiple separate components. Non-limiting examples of fragrance components include aldehydes, ketones, aromatic hydrocarbons, aromatic alcohols and combinations thereof. Specific examples of fragrance ingredients include alpha-hexyl cinnamal, vanillin, citral, eugenol, geraniol, limonene, and citronellol.

Foaming agents are compounds that stabilize foams. Foaming agents increase the propensity of a liquid cleansing composition (104) according to the present specification to form a foam (108), and/or may stabilize a foam (108) by inhibiting the coalescence of bubbles within the foam (108). Certain types of surfactants are capable of acting as foaming agents in a liquid cleansing composition (104); however, not every type of surfactant enhances foam stability. Non-limiting examples of foaming agents include sodium laureth sulfate, cocamidopropyl betaine, cocamidopropyl hydroxysultaine, behenyl betaine, hydroxystearamide MEA, lauramide MEA, myristamide MEA, myristamide DEA, PEG-3 lauramide, PEG-2 lauramine, lauramine oxide, PEG-3 lauramine oxide, and cocamine oxide.

Humectants are hygroscopic substances, or substances which attract water. Humectants may be added to an example of a liquid cleansing composition (104) according to the present specification in order to maintain moisture on the skin to which the liquid cleansing composition (104) is applied. Non-limiting examples of humectants include polyols, urea, panthenol, salts of pyrrolidone carboxylic acid, honey, aloe vera gels, glycerol, sorbitol, glycols, propylene glycol, and butylene glycol.

Emollients are substances that soften the outer layers of the skin. Emollients prevent the evaporation of water in order to preserve the hydration of the skin. Emollients may be added to an example of a liquid cleansing composition (104) according to the present specification in order to preserve moisture on the skin to which the liquid cleansing composition (104) is applied, whereby providing a soft feel to the skin. Non-limiting examples of emollients include PEG-120 methyl glucose dioleate, isopropyl laurate, isopropyl myristate, isopropyl palmitate, isodecyl myristate, isodecyl palmitate, methyl glucose laurate, polyglyceryl-6 tetraoleate, and the like.

Dyes and pigments are compounds which confer color to a liquid cleansing composition (104) or a surface to which a dye or pigment is applied, such as, for example, the surface of a solid particle (106). Dyes and pigments may be added to an example of a liquid cleansing composition (104) according to the present specification in order to imbue the composition with a consumer-acceptable color. Non-limiting examples of dyes and pigments include titanium dioxide, mica, iron oxides, violet 2, red 4, red 6, red 7, red 33, red 40, blue 1, blue 4, yellow 5, yellow 6, yellow 10, orange 4, orange 5, orange 10, vat red 1, vat blue 1, vat blue 4, vat blue 6, vat orange 7, vat violet 2, and combinations thereof.

Conditioning agents are components that act to preserve existing moisture by creating a hydrophobic barrier between the moisturized skin and the air, and may be incorporated into an example of a liquid cleansing composition (104) according to the present specification for this purpose. Non-limiting examples of conditioning agents include the polyquatemium class of polymers, oils, waxes, silicone oils, silicone waxes, fatty alcohols and polyols.

Plant extracts are natural compounds or mixtures of compounds produced in a plant that contain at least one agent that has either a real or perceived benefit to the skin, or to the composition as a whole. The inclusion of some plant extracts may improve consumer acceptance of a liquid cleansing composition (104) on the basis of these benefits, or a consumer preference for naturally produced compositions over synthetically produced compositions. Plant extracts include oils, fragrance ingredients, fatty acids, and/or various other components depending on the extraction methods employed and any subsequent processing that is performed. Non-limiting examples of plant extracts include Prunus Amygdalus dulcis extract, Oenothera biennis extract, Zingiber officinale extract, Jasminum extracts, Lavandula angustifolia extract, Mentha×piperita extract, Rosa extracts, Hypericum perforatum extract, and combinations thereof.

Plant matter is plant material, and may be incorporated into an example of a liquid cleansing composition (104) according to the present specification. Such plant material may provide the solid particles (106) with abrasive properties as exfoliants, fragrance properties, or as a thickener. The incorporation of plant material into liquid cleansing compositions (104) may improve consumer acceptance, which may be based on the perception of the natural qualities of the compositions including plant matter. Non-limiting examples of plant matter include whole flowers, flower petals, stems, seeds, roots, and fruits. Non-limiting examples of plant sources that may provide the plant matter include Citrus plants, Prunus serotina plants, and Zingiber officinale plants.

Essential oils are a particular type of plant extract, which include volatile aroma compounds from the plant from which the essential oil is extracted. The extraction methods used determine the composition of the essential oil. Possible extraction methods include steam distillation, pressure, solvent extraction with organic solvents, solvent extraction with carbon dioxide, and oil extractions, for example. In principle, any type of plant may be used to prepare an essential oil, such as Prunus Amygdalus dulcis essential oil, Oenothera biennis essential oil, Zingiber officinale essential oil, Jasminum essential oils, Lavandula angustifolia essential oil, Mentha×piperita essential oil, Rosa essential oils, and Hypericum perforatum essential oil.

Oils are neutral, nonpolar substances that are viscous liquids at standard ambient temperature and pressure (SATP). Oils may be included in an example of a liquid cleansing composition (104) according to the present specification in order to act as conditioning agents, or to replenish natural oils on the skin. Oils include triglycerides, fatty alcohols, and mineral oils. Mineral oils are oils that are prepared by distillation from crude oil. Non-limiting examples of oils include olive oil, rapeseed oil, paraffinum liquidum, cetyl alcohol, stearyl alcohol, octyldodecanol, sunflower oil, corn oil, palm oil, soybean oil, sunflower oil, and combinations thereof.

Waxes are compounds that have a large fatty content, and are solid at standard ambient temperature and pressure. Waxes may be either hard or malleable at standard ambient temperature and pressure (SATP). For the purposes of the present specification, waxes have a melting point at or above about 45° Celsius (C). Waxes may be included in a liquid cleansing composition (104) according to the present specification in order to improve rheological properties, act as conditioning agents, or to provide other such properties to the composition. Waxes include fatty esters, fatty ethers, hydrocarbons, primary alcohols, secondary alcohols, ketones and aldehydes. Waxes may be alkanes, alkenes, or alkynes, and may be aromatic, anti-aromatic or aliphatic. Waxes may be derived from plants, animals, or crude oil. Non-limiting examples of waxes include cetyl palmitate, lanolin, myristyl palmitate, Carnauba wax, candelilla wax, beeswax, montan wax, paraffin wax, and combinations thereof.

Silicone oils are neutral compounds that are liquids at standard ambient temperature and pressure (SATP). Silicone oils are a type of oil, and may be included in an example of a liquid cleansing composition (104) according to the present specification for the same reasons noted above for oils. Silicone oils may be saturated with hydrocarbon components along a siloxy backbone, which corresponds to the Si—(O—Si)ₙ chain. Silicone oils may also have functional groups incorporated therein. Such functional groups include amines and alcohols. Non-limiting examples of silicone oils include dimethicone, cyclomethicone, dimethiconol, PEG-12 dimethicone, PEG-10 dimethicone, PEG-8 dimethicone, amodimethicone, alkyl methicones, and combinations thereof.

Silicone waxes are silicone compounds that are solid at standard ambient temperature and pressure (SATP). Silicone waxes are a type of waxes, and may be included in an example of a liquid cleansing composition (104) according to the present specification for the same reasons noted above for waxes. For the purposes of the present specification, silicone waxes have a melting point at or above about 45° C. Non-limiting examples of silicone waxes include alkyl methicones, alkyl methicones bearing polyethylene glycol substitutions, crosslinked alkyl methicones, bis-alkyl dimethicones, and combinations thereof.

Chelators are compounds that coordinate metal ions. Chelators may be included in an example of a liquid cleansing composition (104) according to the present specification as antibacterial agents, preservatives, pH regulators, or to provide other such properties to the composition. Non-limiting examples of chelators include natural polyacids (such as citric acid), phosphate salts (such as disodium pyrophosphate), bisphosphonates (such as etridronic acid), aminocarboxylic acids (such as ethylenediaminetetraacetic acid (EDTA) and ethylenediamine-N,N′-disuccinic acid (EDDS)), and combinations and/or salts thereof.

Vitamins are organic compounds that an organism requires in limited quantities, and which the organism that uses the vitamin cannot synthesize from other precursors. Vitamins, or vitamin derivatives, may be included in an example of a liquid cleansing composition (104) according to the present specification as conditioning agents, preservatives, antioxidants, or to improve consumer acceptance of the composition. Non-limiting examples of vitamins include vitamin A (retinol), vitamin B₆ (pyroxidine), vitamin B₇ (biotin), vitamin B₁₂ (cyanocobalamin), vitamin C (ascorbic acid), vitamin E (tocopherols), and vitamin K (phylloquinone). For the purposes of the present specification, “vitamin” also includes derivatives and stereoisomers of vitamins, such as tocopheryl acetate, polyoxypropylene (2) polyoxyethylene (5) tocopherol ether, and isoascorbic acid.

Alkali metal halides are salts of alkali metals and halogen atoms. Alkali metal halides may be included in an example of a liquid cleansing composition (104) according to the present specification as thickeners, ionic strength modulators, or to confer other such properties to the composition. Alkali metal halides are neutral compounds. Non-limiting examples of alkali metal halides include lithium chloride, lithium bromide, sodium chloride, sodium bromide, potassium chloride, potassium bromide, and combinations thereof.

Preservatives are compounds that are added to a liquid cleansing composition (104) according to the present specification to prevent undesirable decomposition over time. Non-limiting examples of preservatives include benzoic acid, benzyl alcohol, phenol, phenoxyethanol, formaldehyde, glyoxal, DMDM hydantoin, cresol, para-hydroxybenzoic acid and esters thereof, chlorhexidine, propionic acid, and salts and/or combinations thereof.

Additives may be incorporated into a liquid cleansing composition (104) according to the present specification at concentrations ranging from 0% to 30% by weight. In one example, a liquid cleansing composition (104) according to the present specification includes additives from 0.01% to 20% by weight. In another example, additives are incorporated into a liquid cleansing composition (104) according to the present specification at concentrations ranging from 0.1% to 10% by weight. The percentages by weight are relative in each case to the total weight of the composition.

As noted above, a liquid cleansing composition (104) according to the present specification do not suspend the solid particles (106) within the liquid cleansing composition (104). Suspending aides are a certain type of thickener, and may be a polymer or copolymer, which is capable of suspending at least one solid particle (106) in solution. Accordingly, a liquid cleansing composition (104) according to the present specification may have a low level of a suspending aide, or may not include any suspending aides. In one example, a liquid cleansing composition (104) according to the present specification contains less than 1.0% by weight, such as less than 0.75% of suspending aides. In a further example, a liquid cleansing composition (104) according to the present specification contains less than 0.5% by weight, such as less than 0.25% by weight of suspending aides. In a still further example, a liquid cleansing composition (104) according to the present specification contains less than 0.1%, such as less than 0.01% of suspending aides. A liquid cleansing composition (104) according to the present specification that contains less than 0.01% of suspending aides is said to be substantially free of suspending aides. However, in some cases, compounds which could be considered suspending aides may also provide a portion of the solid particles (106). Consequently, for the purposes of the present specification, a suspending aide is an agent which provides part of the liquid portion of the liquid cleansing composition (104); when a compound that may be able to act as a suspending aide is provided as a part of the solid particles (106), that compound provides, as a suspending aide, only the portion that dissolves into the liquid cleansing composition (104). Any portion of a suspending aide that remains in solid form, and provides a portion of the solid particles (106), is considered to provide the solid particles (106), and is not a suspending aide for the purposes of the present specification because such a compound in solid form does not act as a suspending aide in a liquid cleansing composition (104) according to the present specification. Non-limiting examples of suspending aides include acrylate/C10-30 alkyl acrylate crosspolymer, acrylates copolymer, acrylates crosspolymer, acrylates crosspolymer-3, acrylates crosspolymer-4, carbomer, cellulose gum, polyacrylate crosspolymer-4, polyacrylate crosspolymer-6, polyacrylate crosspolymer-11, polyacrylate crosspolymer-14, polyacrylic acid, poly C10-30 alkyl acrylate, xanthan gum, guar gum, or combinations thereof.

A liquid cleansing composition (104) according to the present specification also contains a cosmetically suitable carrier, which may also be called a carrier or carrier fluid. Non-limiting examples of cosmetically suitable carriers include water and organic solvents, such as C₁-C₆ alcohols, glycols, glycerol, and the like. Specific cosmetically suitable carriers include, either alone or in combination, water, ethanol, n-propanol, isopropanol, n-butanol, isobutanol, n-pentanol, 2-pentanol, 3-pentanol, polyethylene glycols such as PEG-4, PEG-6, PEG-8, PEG-10, and PEG-12, propylene glycol, polypropylene glycols such as PPG-3, PPG-6, PPG-9 and PPG-12, butylene glycol, polybutylene glycol, and glycerol. In one example, a liquid cleansing composition (104) according to the present specification includes water as the cosmetically suitable carrier. In another example, a liquid cleansing composition (104) according to the present specification includes a mixture of water and propylene glycol as the cosmetically suitable carrier. In a further example, a liquid cleansing composition (104) according to the present specification includes a C₁-C₆ alcohol, such as ethanol or isopropanol as the cosmetically suitable carrier. In a still further example, a liquid cleansing composition (104) according to the present specification includes a mixture of water, glycerol, and 1,2-propanediol as the cosmetically suitable carrier.

Viscosity is a measurement of the resistance of a fluid to gradual deformation due to physical stress. The viscosity of a liquid cleansing composition (104) according to the present specification is measured on the liquid form of a liquid cleansing composition (104), excluding the solid particles (106). For example, if the liquid cleansing composition (104) is a foaming composition, the viscosity is measured prior to formation of a foam (108). A liquid cleansing composition (104) according to the present specification may have a viscosity in the range of 0 to 4,000 centipoise (cps), such as from 0 to 3,000 cps. In one example, a liquid cleansing composition (104) according to the present specification has a viscosity in the range of 0 to 1,000 cps. In a further example, a liquid cleansing composition (104) according to the present specification has a viscosity in the range of 0 to 500 cps. In a still further example, a liquid cleansing composition (104) according to the present specification has a viscosity in the range of 0 to 200 centipoise.

A liquid cleansing composition (104) according to the present specification is provided in a container (102). Such a container (102) may be transparent, in order to allow observation of visual effects of the motion of the solid particles (106) upon agitation of the liquid cleansing composition (104) in the container (102). Transparency is measured using ASTM International standard D-1003 (2013 revision) using light within the visible spectrum. Light within the visible spectrum is light that has a wavelength from 400 nanometers (nm) to 700 nm, inclusive. ASTM International Standard D-1003 provides measurements of transparency that are in units of percent transmittance, which represents the percentage of incident light that is transmitted through the material. This measurement is referred to as the degree of light transmission of the material. For the purposes of the present specification, a material is said to be transparent if the percent transmittance of light within the visible spectrum, as measured by ASTM International Standard D-1003, is at least 60%. In one example, the percent transmittance of a transparent material according to the present specification is at least 85%, as measured by ASTM International Standard D-1003. The transparency of a container (102) is a function of both the transparency of the material from which the container (102) is made, as well as the thickness of the material. Increasing the thickness of a transparent material can cause the material to appear less transparent.

ASTM International standard D-1003 (2013 revision) may also be used to measure the haze of a material. Haze is a measurement of the scattering of light through a material, and is expressed as a percentage of light that is scattered so that its direction deviates by more than a specified angle from the direction of the incident beam of light. The angle is specified in accordance with ASTM International standard D-1003. As with the transparency measurements, haze measurements are conducted with light in the visible spectrum. A lower value of haze indicates that the material being tested scatters a smaller fraction of the light. In one example, a container (102) according to the present specification is made from a material with a haze between 0% and 50%, such as from 0% to 30%, or from 0% to 15%.

A container (102) according to the present specification may have a thickness that allows a user to perceive the container (102) as transparent. In one example, a container (102) according to the present specification has a wall thickness that ranges from 0.1 mm to 3.0 mm. In another example, a container (102) according to the present specification has a wall thickness that ranges from 0.2 mm to 2.0 mm. In a further example, a container (102) according to the present specification has a wall thickness that ranges from 0.4 mm to 1.5 mm.

A container (102) according to the present specification may also have a smooth or roughened surface. For the purposes of the present specification, surface roughness (R_{z}) is measured using ISO 4287 (DIN 4728, 2009 revision). The surface roughness, R_{z}, is the arithmetic mean of the absolute value of the profile departure with a provided length. In one example, a container (102) according to the present specification has a surface that is, or is perceived as, smooth. In another example, a container (102) according to the present specification has a surface roughness, R_{z} of between 0.1 micrometers (μm) and 50 μm as measured by ISO 4287. In a further example, a container (102) according to the present specification has a surface roughness, R_{z} of between 0.1 μm and 20 μm as measured by ISO 4287.

A container (102) according to the present specification is said to be transparent if the main body of the container (102) is made from a transparent material of a thickness such that the contents of the container (102) are visible through the main body of the container (102). The transparency of a container (102) is considered separately from the transparency of the lever (110), foam generation chamber (112), dip tube (114) and pre-filter (116).

Accordingly, a container (102) according to the present specification may be made from a plastic that is transparent. A transparent container (102) according to the present specification may be clear, or may contain a quantity of dye sufficient to provide a color to the container (102). In one example, a container (102) according to the present specification is a clear transparent container (102). In another example, a container (102) according to the present specification is a green transparent container (102). In a further example, a container (102) according to the present specification is a blue transparent container (102). There is no intention to limit the choice of dyes that may be used in a container (102) according to the present specification; any color within the visible light spectrum may be suitable.

Containers (102) made from certain materials are also be more readily reused or recycled than containers (102) made from other materials. Accordingly, it may be suitable to use a material for the container (102) that is both transparent and readily reused or recycled in order to both decrease the ecological impact of such a container (102), as well as increase consumer appeal as a consequence of the decreased ecological impact.

A container (102) may be made of any suitable material, for example glass or plastic. A glass container (102) may have consumer appeal because the container (102) may be reusable. A glass container (102) may also be recyclable; however, this depends on the type of glass used for the container (102). For example, container glass is readily recycled, while heat resistant glass is more difficult to recycle. A plastic container (102) may be more economical to produce than a glass container (102), and may also be recyclable. Certain types of plastic, for example polyvinyl chloride (PVC), are recycled with high efficiency. Other types of plastic, for example polystyrene (PS), are more difficult to recycle.

Non-limiting examples of materials for a container (102) according to the present specification include borosilicate glass, container glass, polypropylene (PP), polyethylene (PE), polyvinyl chloride (PVC), polycarbonate (PC), polystyrene (PS), and polyesters such as polyethylene terephthalate (PET), polyethylene terephthalate glycol-modified (PET-G), polycyclohexylenedimethylene terephthalate (PCT), polycyclohexylenedimethylene terephthalate glycol-modified (PCTG), polyhydroxybutyrate (PHB), and combinations thereof. In one example, a container (102) according to the present specification is prepared from a plastic resin that has been extruded, granulated or stretched. In another example, a container (102) according to the present specification is made from polypropylene, which may be a random copolymer resin or a block copolymer resin.

A container (102) according to the present specification may also include an agent that blocks or absorbs ultraviolet (UV) light. The inclusion of an agent that blocks or absorbs UV light prevents or reduces the degradation of light sensitive materials in a liquid cleansing composition (104). UV light is light that has a wavelength in the range of 280 nm to 400 nm, inclusive. Any agent that blocks or absorbs UV light may be suitable. In one example, a container (102) according to the present specification includes as a UV absorbing agent avobenzone, octyl methoxycinnamate, ortho-hydroxy benzophenones, ortho-hydroxy phenyl triazines, and ortho-hydroxy phenyl hydrazines. In a further example, UV absorbers and/or Hindered Amine Light Stabilizers (HALS) are included in the container (102) at a concentration of 0.1% to 2% by weight, such as from 0.5% to 1% by weight. In another example, a container (102) according to the present specification uses UV blockers, such as titanium dioxide or zinc oxide. In one example, a UV absorber, HALS or UV blocker is included in a container (102) according to the present specification, and reduces the intensity of incident UV light by an amount of from 0% to 25% of the intensity of the incident light, such as from 0% to 15% of the intensity of the incident light, or from 0% to 5% of the intensity of the incident light. In a further example, a UV absorber, HALS or UV blocker is included in a container (102) according to the present specification, and reduces the intensity of incident UV light by an amount of from 5% to 25%, such as from 5% to 15%, of the intensity of the incident light. In a still further example, the inclusion of a UV absorber, HALS or UV blocker does not affect the transmission of light in the visible spectrum.

A container (102) according to the present specification may be made by any suitable method. In one example, a container (102) according to the present specification is made by extrusion blow molding (EBM), injection stretch blow molding (ISBM), injection blow molding (IBM), or injection molding and thermoforming. The choice of method for preparing a container (102) may depend on the material(s) chosen for the container (102) and/or production cost.

A container (102) according to the present specification may assume any suitable shape. According to one example, a container (102) according to the present specification also includes a foam generation chamber (112), lever (110), and dip tube (114). In this example, the dip tube (114) is equipped with a pre-filter (116). In this example, the liquid cleansing composition (104) is dispensed as a foam (108). In a further example, a container (102) according to the present specification includes a lever (110) and a dip tube (114), but does not contain a foam generation chamber (112); in this example, the liquid cleansing composition (104) is dispensed as a liquid.

If a foam generation chamber (112) is included, the foam generation chamber (112) may be provided as a single unit with the lever (110) and the dip tube (114), and may include a pre-filter (116) on the dip tube (114). A foam generation chamber (112) may operate by any suitable apparatus for generation of foam (108). In one example, a foam generation chamber (112) operates by passing gas through a mesh in the presence of the liquid cleansing composition (104), causing bubbles to form in the liquid cleansing composition (104), such that the liquid cleansing composition (104) takes the form of a foam (108). According to one example, the gas employed is air taken in from the environment outside the container (102). The actuation of the lever (110) may provide the compressive force that drives the gas through the mesh within the foam generation chamber (112). The dip tube (114) draws the liquid cleansing composition (104) into the foam generation chamber (112). The lever (110), dip tube (114) and/or foam generation chamber (112) may be equipped with a one-way valve, so that the foam generation chamber (112) refills with both the liquid cleansing composition (104) and gas (which, in some examples, is air from the outside environment) when the lever (110) is released.

A liquid cleansing composition (104) that is dispensed through a foam generation chamber (112) is in the form of a foam (108). Such a foam (108) includes bubbles which are filled with a gas, which may be air from the surrounding environment. According to one example, a foam (108) contains at least 50% by volume of gas. In a further example, a foam (108) contains at least 60% by volume of gas, such as at least 70% by volume of gas. In a still further example, a foam (108) contains at least 75% by volume of gas, such as at least 80% by volume of gas.

As noted above, a liquid cleansing composition (104) according to the present specification may be dispensed with the solid particles (106), or may be dispensed without the solid particles (106), and may be dispensed as a foam (108), or as a liquid. In one example, solid particles (106) are filtered out of a liquid cleansing composition (104) according to the present specification by the use of a pre-filter (116), and the liquid cleansing composition (104) is dispensed as a foam (108) that does not contain solid particles (106). In another example, solid particles (106) are contained in the foam (108) of the liquid cleansing composition (104) according to the present specification that is dispensed from a container (102). In a further example, a liquid cleansing composition (104) according to the present specification is dispensed as a liquid that does not contain solid particles (106). In a still further example, a liquid cleansing composition (104) according to the present specification is dispensed as a liquid that does include solid particles (106). If a liquid cleansing composition (104) according to the present specification is dispensed as a liquid, without foam generation, it may be advantageous for the liquid cleansing composition (104) to have a higher viscosity, such as, for example, from 500 to 3,000 centipoise (cps), or from 1,000 to 2,000 cps.

A consumer product according to the present specification may include a liquid cleansing composition (104) that includes solid particles (106), held within a container (102) that is equipped with a lever (110), a foam generation chamber (112), and a dip tube (114), which is optionally equipped with a pre-filter (116). The densities of the liquid cleansing composition (104) and the solid particles (106) may be chosen so that the solid particles (106) sink within the liquid cleansing composition (104) when the container (102) is at rest. In one example, the rate at which the solid particles (106) sink is slow, so that upon agitation of the liquid cleansing composition (104), the solid particles (106) move within the liquid cleansing composition (104) to create a visual effect. For the purposes of the present specification, the rate at which the solid particles (106) sink is said to be slow if the terminal velocity of the solid particles (106) in the liquid cleansing composition (104) is less than 5 centimeters per second. Such a visual effect may involve the solid particles (106) swirling within the container (102), gradually falling, and/or creating the appearance of falling snow, which may be white, red, orange, pink, blue, green, black, copper, silver, gold, goniochromatic, having a metallic reflection of incident light, or combinations thereof.

Solid particles (106) according to the present specification may be chosen so as to achieve a pleasant visual effect. In one example, the solid particles (106) have a specific gravity that is slightly greater than the liquid cleansing composition (104) without the solid particles (106), such as from 1.01 to 1.3. The shape of the solid particles (106) may also be chosen in order to enhance the visual effect, or to affect the rate at which the solid particles (106) sink within the liquid cleansing composition (104). In one example, the solid particles (106) are discs, which fall slower than a sphere due to greater frictional force between the liquid cleansing composition (104) and the solid particles (106); this frictional force is referred to as the Stokes' drag. According to one example, solid particles (106) according to the present specification are chosen so as to have a terminal velocity within the liquid cleansing composition (104) of less than or equal to 5 centimeters per second (cm/s). In another example, solid particles (106) according to the present specification are chosen so as to have a terminal velocity within the liquid cleansing composition (104) of less than or equal to 4 cm/s, such as less than or equal to 3 cm/s. In a further example, solid particles (106) according to the present specification are chosen so as to have a terminal velocity within the liquid cleansing composition (104) of less than or equal to 2 cm/s, for example less than or equal to 1 cm/s. For a spherical particle, the terminal velocity can be derived from Stokes' law by setting the Stokes' drag to be equal to the gravitational force acting on the solid particles (106). Thus, for a spherical particle, the terminal velocity may be expressed as

$$v = ( \frac{2}{9} ) \cdot ( \frac{( {ρ}_{106} - {ρ}_{104} )}{{μ}_{104}} ) \cdot g \cdot {( {r}_{106} )}^{2}$$

where ν is the terminal velocity, ρ₁₀₆ is the density of the solid particles (106), ρ₁₀₄ is the density of the liquid cleansing composition (104), μ₁₀₄ is the viscosity of the liquid cleansing composition (104), expressed in units of Pascal-seconds (Pa·s), g is the gravitational acceleration, and r₁₀₆ is the radius of the solid particle (106). The above equation also provides an approximation of the terminal velocity for non-spherical solid particles (106), by considering either the smallest sphere capable of enclosing the solid particle (106), or a sphere of equal volume to the volume of the solid particle (106).

A consumer product according to the present specification may include a pendant (118), which may be attached to a dip tube (114). Such a pendant (118) may be any shape, size or color, and may be included for a visual effect. The inclusion of a pendant (118) may enhance the visual effect provided by the solid particles (106).

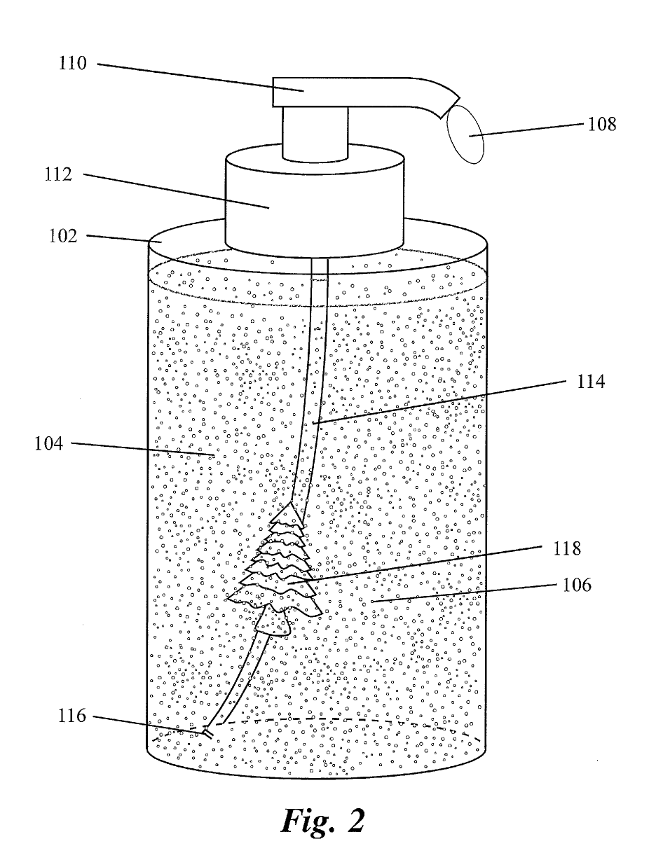

FIG. 2 is a diagram of a consumer product containing a cleansing composition (104) that includes solid particles (106) in a dispersed state, according to an example of the principles described herein. To achieve the dispersed state shown in FIG. 2, the container (102), and the liquid cleansing composition (104) therein, have been agitated. In the diagram shown in FIG. 2, the solid particles (106) are dispersed throughout the liquid cleansing composition (104), and are beginning the process of re-settling on the bottom of the container (102); the solid particles (106) may achieve a terminal velocity in the settling process, which is approximated by the above equation. As in FIG. 1, the container (102) holds a liquid cleansing composition (104) and solid particles (106). The liquid cleansing composition (104) shown in FIG. 2 is dispensed as a foam (108) upon actuation of the lever (110), which produces foam (108) in the foam generation chamber (112). The foam generation chamber (112) of FIG. 2 is supplied with the liquid cleansing composition (104) through the dip tube (114), which is equipped with a pre-filter (116). The dip tube (114) shown in FIG. 2 is also equipped with a pendant (118).

### EXAMPLES

The examples that follow indicate liquid cleansing compositions with visual particles that may be prepared in accordance with the present specification. Unless otherwise indicated, the stated quantities are percentages by weight.

### Example 1

### Foaming Liquid Hand Soap with Solid Particles

| Formulation Example 1 | Wt. % |
|---|---|
| Anionic Surfactant(s) | 15-30 |
| Zwitterionic Surfactant(s) | 5-10 |
| Humectant(s) | 2-8 |
| Emollient(s) | 0-2 |
| Chelator(s) | 0-1 |
| Preservative(s) | 0-1 |
| Fragrance | 0-1 |
| Synthetic Polymer(s) | 0-3 |
| Solid Particles (FIG. 1, 106) | 1-5 |
| Carrier (Water) | Q.S. 100 |

### Example 2

### Foaming Antibacterial Hand Soap with Solid Particles

| Formulation Example 2 | Wt. % |
|---|---|
| Amphoteric Surfactant(s) | 0.5-5 |
| Cationic Surfactant(s) | 1-4 |
| Nonionic Surfactant(s) | 0-3 |
| Humectant(s) | 1-5 |
| Emollient(s) | 0.1-3 |
| Chelator(s) | 0-1 |
| pH Adjuster(s) | 0-3 |
| Preservative(s) | 0-1 |
| Fragrance | 0-2 |
| Thickening Agent(s) | 0-1 |
| Antibacterial Agent(s) | 0.001-3 |
| Dye(s) | 0-2 |
| Solid Particles (FIG. 1, 106) | 1-5 |
| Carrier (Water) | Q.S. 100 |

### Example 3

### Foaming Hand Sanitizer

| Formulation Example 3 | Wt. % |
|---|---|
| Cationic Surfactant(s) | 0.2-4 |
| Humectant(s) | 0.1-4 |
| Skin Conditioner(s) | 0.1-4 |
| Emollient(s) | 0-2 |
| Fragrance | 0-2 |
| Solid Particles (FIG. 1, 106) | 1-5 |
| Carrier (Ethanol/Water Mixture) | Q.S. 100 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A composition for cleansing skin, comprising, in a cosmetically suitable carrier:
at least one surfactant to cleanse skin; and
at least one solid particle;
in which:
the cleansing composition is dispensed as a foam; and
the at least one solid particle is visible to the human eye without the aid of a magnifying lens or optical device.

2. The cleansing composition of claim 1, in which the at least one solid particle has a specific gravity of greater than 1, relative to the cosmetically suitable carrier.

3. The cleansing composition of claim 1, in which the viscosity of the cleansing composition, excluding the solid particles, is between 0 and 3,000 centipoise (cps).

4. The cleansing composition of claim 1, further comprising a thickening agent.

5. The cleansing composition of claim 1, in which the cleansing composition is contained within a vessel, and the solid particles settle to a bottom of the vessel.

6. The cleansing composition of claim 5, in which the solid particles have an average terminal velocity in the cleansing composition of less than or equal to 3 centimeters per second (cm/s).

7. The cleansing composition of claim 1, further comprising an additive ingredient selected from a natural polymer, a synthetic polymer, a semi-synthetic polymer, an antibacterial agent, a fragrance, a foaming agent, a humectant, an emollient, a dye, a pigment, a conditioning agent, a plant extract, plant matter, an essential oil, an oil, a wax, a silicone oil, a silicone wax, a chelator, a vitamin, a vitamin derivative, an alkali metal halide, and combinations thereof.

8. The cleansing composition of claim 1, in which the at least one solid particle is sparingly soluble in the cosmetically suitable carrier.

9. The cleansing composition of claim 1, in which the at least one surfactant comprises cocamidopropyl betaine.

10. The cleansing composition of claim 1, in which the cosmetically suitable carrier comprises water.

11. The cleansing composition of claim 10, in which the cosmetically suitable carrier further comprises an organic solvent.

12. A consumer product for cleansing skin, comprising:
a container; and
a cleansing composition, comprising:
a cosmetically suitable carrier;
at least one surfactant to cleanse skin;
at least one solid particle;
in which:
the cleansing composition has a viscosity, excluding the solid particles, of between 0 and 3,000 centipoise (cps); and
the at least one solid particle sinks within the cleansing composition when the container is at rest;
the cleansing composition contains less than 0.1% of a suspending polymer.

13. The consumer product of claim 12, in which the container is transparent.

14. The consumer product of claim 12, in which the viscosity of the cleansing composition is between 0 cps and 500 cps.

15. The consumer product of claim 12, in which the cleansing composition further comprises an additive ingredient selected from a natural polymer, a synthetic polymer, a semi-synthetic polymer, an antibacterial agent, a fragrance, a foaming agent, a humectant, an emollient, a dye, a pigment, a conditioning agent, a plant extract, plant matter, an essential oil, an oil, a wax, a silicone oil, a silicone wax, a chelator, a vitamin, a vitamin derivative, an alkali metal halide, and combinations thereof.

16. The consumer product of claim 12, in which the at least one solid particle is dispensed with the cleansing composition.

17. The consumer product of claim 12, in which the container comprises a dip tube equipped with a pre-filter, and the at least one solid particle remains within the container when the cleansing composition is dispensed from the container.

18. The consumer product of claim 12, in which agitation of the container causes the at least one solid particle to be dispersed throughout the cleansing composition, and when the agitation ceases, the settling of the at least one solid particle to a bottom of the container takes at least 2 seconds.

19. A consumer product for cleansing the skin, comprising:
a transparent plastic container comprising a dispenser unit;
a cleansing composition comprising:
a cosmetically suitable carrier;
at least one surfactant, comprising a zwitterionic surfactant; and
at least one solid particle;
in which:
the cleansing composition is dispensed from the transparent plastic container in the form of a foam which contains at least 50% by volume of gas;
the at least one solid particle is sparingly soluble in the cleansing composition; and
the at least one solid particle has a specific gravity, relative to the cosmetically suitable carrier, of greater than or equal to 1.

20. The consumer product of claim 19, in which the cleansing composition, excluding the solid particles, has a viscosity of from 0 to 500 centipoise.
